SEQUENCE LISTING (12 sequences)

SEQ ID NO: 1 - DNA, 2593 bp, Artificial Sequence
  Synthetic- Universal construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttcatccggcaaacaaaccaccgctggtagcggtggtttttttgtttgcaagcagcagattacgcgcagaaaaaaaggatctcaagaagatcctttgatcttttctagggataacagggtaatgtggaacgaaaactcacgttaagggattttggtcatgctcttcgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatgctcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtagggataacagggtaatcagacagttttattgttcatgatgatatatttttatcttgtgcagctcttcatgtaacatcagagattttgagacacgggccagagctgca

SEQ ID NO: 2 - DNA, 56 bp, Artificial Sequence
  Synthetic- Protelomerase TelN recognition sequence
tatcagcacacaattgcccattatacgcgcgtataatggactattgtgtgctgata

SEQ ID NO: 3 - DNA, 1356 bp, Artificial Sequence
  Synthetic-Target sequence 1
ggtagctaggagttccaggactcagtttcccctttgagcctcctttagcgactaaagcttgaagccccacgcatctcgactctcgcgcacaccgcccttgttgggctcaggggcggggcgccgcccccggaagtacttccccttaaaggctggggcctgccggaaatggcgcagcggcagggaggggctcttcacccagtccggcagttgaagctcggcgctcgggttacccctgcagcgacgccccctggtcccacagataccactgctgctcccgccctttcgctcctcggccgcgcaatgggcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaaccggtgctggaagtggtacacgcgacgacgagtacgactacctctttaaaggtgaggccatgggctctcgcactctacacagtcctcgttcggggacccgggccactcccggtggaccctcgtgccggccacccctgcactgatataggcctccctcagcccttcctttttgtgcggttccgtctcctacccagctcagcctcttctcccccgctcagacaggggtccccatcacatgccgctctctgagcgacctctccataggccttcgctggcctcagagcccctccctgcgtgtccttcccctggcggactgccttctcccacatcgt

SEQ ID NO: 4 - DNA, 300 bp, Artificial Sequence
  Synthetic-RAB11a left homologous arm
ggtagctaggagttccaggactcagtttcccctttgagcctcctttagcgactaaagcttgaagccccacgcatctcgactctcgcgcacaccgcccttgttgggctcaggggcggggcgccgcccccggaagtacttccccttaaaggctggggcctgccggaaatggcgcagcggcagggaggggctcttcacccagtccggcagttgaagctcggcgctcgggttacccctgcagcgacgccccctggtcccacagataccactgctgctcccgccctttcgctcctcggccgcgca

SEQ ID NO: 5 - DNA, 300 bp, Artificial Sequence
  Synthetic-RAB11a right homologous arm
ggaaccggtgctggaagtggtacacgcgacgacgagtacgactacctctttaaaggtgaggccatgggctctcgcactctacacagtcctcgttcggggacccgggccactcccggtggaccctcgtgccggccacccctgcactgatataggcctccctcagcccttcctttttgtgcggttccgtctcctacccagctcagcctcttctcccccgctcagacaggggtccccatcacatgccgctctctgagcgacctctccataggccttcgctggcctcagagcccctccctgcgt

SEQ ID NO: 6 - DNA, 1468 bp, Artificial Sequence
  Synthetic-Target sequence 1+TelN recognition sequence
tatcagcacacaattgcccattatacgcgcgtataatggactattgtgtgctgataggtagctaggagttccaggactcagtttcccctttgagcctcctttagcgactaaagcttgaagccccacgcatctcgactctcgcgcacaccgcccttgttgggctcaggggcggggcgccgcccccggaagtacttccccttaaaggctggggcctgccggaaatggcgcagcggcagggaggggctcttcacccagtccggcagttgaagctcggcgctcgggttacccctgcagcgacgccccctggtcccacagataccactgctgctcccgccctttcgctcctcggccgcgcaatgggcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaaccggtgctggaagtggtacacgcgacgacgagtacgactacctctttaaaggtgaggccatgggctctcgcactctacacagtcctcgttcggggacccgggccactcccggtggaccctcgtgccggccacccctgcactgatataggcctccctcagcccttcctttttgtgcggttccgtctcctacccagctcagcctcttctcccccgctcagacaggggtccccatcacatgccgctctctgagcgacctctccataggccttcgctggcctcagagcccctccctgcgtgtccttcccctggcggactgccttctcccacatcgttatcagcacacaattgcccattatacgcgcgtataatggactattgtgtgctgata

SEQ ID NO: 7 - DNA, 318 bp, Artificial Sequence
  Synthetic-TRAC left homologous arm
tttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccaagcttgggagttccg

SEQ ID NO: 8 - DNA, 304 bp, Artificial Sequence
  Synthetic-TRAC right homologous arm
gtgtaccagctgagagactctaagtccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggcca

SEQ ID NO: 9 - DNA, 1885 bp, Artificial Sequence
  Synthetic-Target sequence 2
tttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccaagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtgtaccagctgagagactctaagtccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggcca

SEQ ID NO: 10 - DNA, 1997 bp, Artificial Sequence
  Synthetic-Target sequence 2+TelN recognition sequence
tatcagcacacaattgcccattatacgcgcgtataatggactattgtgtgctgatatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccaagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtgtaccagctgagagactctaagtccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccatatcagcacacaattgcccattatacgcgcgtataatggactattgtgtgctgata

SEQ ID NO: 11 - RNA, 100 nt, Artificial Sequence
  Synthetic-sgRNA
agagucucucagcugguacaguuuuagagcuagaaauagcaaguuaaaauaaggcuaguccguuaucaacuugaaaaaguggcaccgagucggugcuuuu

SEQ ID NO: 12 - DNA, 2579 bp, Artificial Sequence
  Synthetic-pUC57-KanR
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

(19) United States
(12) Patent Application Publication
(10) Pub. No.: US 2024/0084285 A1
(43) Pub. Date: Mar. 14, 2024

(54) METHOD FOR PRODUCING TARGET DNA SEQUENCE AND CLONING VECTOR

(71) Applicant: NANJING GENSCRIPT BIOTECH CO., LTD., (CN)

(72) Inventors: Lumeng YE, Nanjing, Jiangsu (CN); Haiye SUN, Nanjing, Jiangsu (CN); Qian GAO, Nanjing, Jiangsu (CN); Juan CHEN, Nanjing, Jiangsu (CN)

(21) Appl. No.: 18/262,420

(22) PCT Filed: Aug. 6, 2021
(86) PCT No.: PCT/CN2021/111152

§ 371 Date: Jul. 21, 2023

(30) Foreign Application Priority Data
Jan. 22, 2021 (CN) 202110087186.9

(51) Int. Cl. C12N 15/10; C12N 9/12; C12N 9/22; C12N 15/63

(52) U.S. Cl. CPC C12N 15/1003; C12N 9/1276; C12N 9/22; C12N 15/63; C12N 2800/80

(57) ABSTRACT

Provided is a method for producing a target DNA sequence and a cloning vector. The method includes the step of amplifying and extracting a DNA construct in a host cell, and a three-step thermostatic enzyme reaction step of protelomerase-IIS type restriction endonuclease and/or meganuclease-DNA exonuclease catalysis, wherein the construct is autonomously replicated and contains (a) one or more IIS type restriction endonuclease and/or meganuclease recognition sequences; (b) the target DNA sequence; and (c) protelomerase recognition sequences at lateral wings of two ends of the target DNA sequence.

### BACKGROUND

### Technical Field

The present application relates to the field of bioengineering, in particular to a method for producing a target DNA sequence and a cloning vector.

### Related Art

After more than 30 years of development, a gene therapy and a cell therapy based on gene editing are sufficiently developed. Especially, precise gene editing based on nuclease (zinc finger nuclease. TAL nuclease and CRISPR nuclease) and a delivery mode being independent of viruses make the safer and more-efficient gene therapy being independent of viruses to become possible. In an early stage, a plasmid is used for delivering and expressing a target protein gene as for treatment of a gene defective type genetic disease so as to perform compensation. When large fragment genes are knocked in based on homologous recombination, the plasmid or a linearized plasmid fragment is also used as an editing template. Practice has proven that application of the plasmid in gene editing has many shortcomings and potential risks: 1) due to containing a redundant vector skeleton sequence of a non-target fragment, greater cytotoxicity based on double-stranded DNA during in-vivo or immune cell delivery; 2) the redundant skeleton sequence on the plasmid is mainly a replication origin site and antibiotic resistance genes, these genes are possibly polluted by normal microbial flora of human when being used for the gene therapy, and moreover, the sequence with the bacterial source generally contains a CpG island, is prone to being methylated in a plasmid replication process, and generates powerful immunogenicity during application; and 3) during homologous recombination repair, the blunt-end double-stranded DNA prepared on the basis of a PCR amplification method is adopted as a repair template, which possibly results in non-homologous recombination repair end joining, resulting in the edited sequence containing a repeated homologous arm region, and damaging an original design.

Aiming at solving the above problems, there is research trying to separate a target fragment from the plasmid in a double enzyme digestion mode. However, the method is difficult to realize on large-scale fragment separation and purification and can only perform small-scale purification through agarose gel electrophoresis. Moreover, the method performs color development on the DNA fragment depending on a DNA dye, and whether the embedded DNA dye can be completely cleared or not is also a problem difficult to verify. Although in 2016, Slavcev et al reported a fermentation technology of the target fragment double-stranded DNA realized based on bacteria in-vivo recombination, its result has shown that due to an incomplete recombination efficiency, a product purified after thallus lysis will have pollution of DNA impurities such as a bacterial genome and an original plasmid, and a high-purity end product is also separated depending on the agarose gel electrophoresis. That is, the method still has the above problems of being difficult to scale up and the toxic DNA dye in industrial production.

Another try for directly obtaining the target fragment is to prepare a large amount of the double-stranded DNA through PCR amplification. However, the DNA polymerase used by PCR is inferior to that of a DNA replication system in bacterial bodies in sequence fidelity. During large-scale preparation, the cost for purchasing a large amount of high-fidelity DNA polymerase is very high. When the large fragment is amplified, the yield of the PCR is slightly decreased, and non-full-length fragment pollution possibly occurs. The critical question is that when a milligram-level double-stranded DNA pure product is needed, a large amount of PCR needs to be performed to collect hundreds or even thousands of products of PCR reactions together, which is difficult to conform to a GMP norm. In order to solve the problem, the Touchlight Genetics company in United Kingdom develops a double-stranded DNA in-vitro thermostatic index amplification method based on rolling circle amplification (RCA). However, when a target product is used as a template for gene knock-in, the in-vitro replication method will have a risk of increasing DNA sequence mutation due to lack of a DNA replication error-correcting mechanism in the bacterial body. Particularly, when the target sequence contains an extremely high proportion of a GC sequence, a hairpin structure and a repetitive sequence, it is difficult to ensure by in-vitro replication based on the DNA polymerase that losing will generally not occur in a high-difficulty sequence region. Moreover, the overall in-vitro replication process is in index replication and is random, and when a target sequence monomer is cut and released from a compound after the reaction is completed, the product will have the impurities mixed with incomplete fragment product and difficult to remove.

A simple and efficient method for preparing the target DNA sequence is still needed.

### SUMMARY

The present application provides a method for producing a target DNA sequence and a cloning vector. The method and the cloning vector have no special limitation for the to-be-produced target DNA sequence and therefore are a universal method and the cloning vector suitable for various DNA sequences. Moreover, the method and the cloning vector can be configured to efficiently produce the high-purity target DNA sequence on a large scale. The method uses a DNA construct containing a protelomerase recognition sequence and an HS type restriction endonuclease and/or meganuclease recognition sequence, the DNA construction bodies are massively produced through an intracellular vector amplification process (for example, through plasmid transformation and extraction method), and then a high-purity target DNA fragment may be obtained through three steps of thermostatic enzyme reaction of protelomerase-HS type restriction endonuclease and/or meganuclease-DNA exonuclease. An end product may be subjected to alcohol (for example, ethanol) precipitation concentration and is easy to prepare on a large scale. An overall preparation and purification process does not involve a DNA dye. The end product is derived from in-vivo replication through host cells, the sequence is high in accuracy, and sequence error, losing or mutation due to containing a high-difficulty sequence in the target sequence is avoided.

Cloning Vector

The present application provides a universal cloning vector configured to produce the target DNA sequence or to construct a DNA construct described hereinafter. The universal cloning vector is an autonomously replicating vector and contains: (a) one or more HS type restriction endonuclease and/or meganuclease recognition sequences, and (b) multiple cloning sites.

In some implementations, the cloning vector is selected from: a plasmid, a cosmid, phage or viruses (for example, retrovirus, adeno-associated virus, lentivirus, rhabdovirus and adenovirus). The cloning vector may contain a replication origin. The cloning vector may contain one or more restriction endonuclease recognition sites or multiple cloning sites configured to insert a foreign DNA sequence (for example, the target DNA sequence with a lateral wing connected with a protelomerase recognition sequence), and/or a selective marker gene (for example, an antibiotic resistance gene and a ccdB gene) configured to recognize and select cells transformed by the cloning vector.

In some implementations, the cloning vector contains two or more, for example, 3, 4, 5, 6, 7, 8, 9, 10 or more HS type restriction endonuclease and/or meganuclease recognition sequences. In some embodiments, the cloning vector contains 3 or more HS type restriction endonuclease and/or 2 or more meganuclease recognition sequences. In one specific embodiment, the cloning vector includes 5 HS type restriction endonuclease and/or 2 meganuclease recognition sequences.

The IIS type restriction endonuclease used herein includes but is not limited to:
AlwI, Bed, BsmAl, EarI, Bmrl, BsaI, BsmBI, Faul, HpyAV, Mn1I, Sapl, BbsI, BciVI, HphI, IMboH, BfuaI, BspM1, SfaNI, Bbvl, Ecif, FokI, BceAI, Bstnlq, BtgZI, Bprnl, BpuEI, BsgI, AOKI, Alw26I, Bst6I, BsrDI, BstJvlAI, Eam11041, Ksp632I, Ppsl, SchI, Bfii, Bso3 I, BspTNI, BspQI, Eco31I. Esp3I, Faul, Sarni, Bful, Bpil, BpuAI,
BstV2I, Acc361, Lwei, Aarl, BseMll, TspDTI, BseXI, BstVll, Eco57I, Eco57MI, GsuI, Psrl, or MmeI. In some implementations, the HS type restriction endonuclease is selected from one or any combination of the following: BbsI, BsaI, BstnIll, BspQI, BsrDI, EarI, ITIgaI and SfaNI. A method for preparing and using the HS type restriction endonuclease is conventional, and many HS type restriction endonucleases can be obtained commercially. The “HS type restriction endonuclease recognition sequence” is a sequence capable of being recognized and cut by the corresponding HS type restriction endonuclease, is determined according to the specific used HS type restriction endonuclease and is known in the art. In one embodiment, the HS type restriction endonuclease contains BspQI with the recognition sequence of GCTCTTC.

The term “meganuclease” used herein is an endonuclease subtype with a rare nick greater than 12 bp of a double-stranded DNA target sequence. The meganuclease is generally dimeric enzyme, is also called homingendonuclease (HE), and can be divided into five families: LAGLIDADG, HNH, His-Cys box and TO-(D/E)XK according to the sequence and a structural motif. Structural data is available for at least one member of each family. In some implementations, the meganuclease is selected from any one or any combination of the following: I-Seel, I-CreI, I-DmoI, I-OnuI, I-GzeMII, I-LtrWI and I-Sma.MI. A method for preparing and using the meganuclease is conventional, and many meganucleases can be obtained commercially. The “meganuclease recognition sequence” is a sequence capable of being recognized and cut by the corresponding meganuclease, is determined according to the specific used meganuclease and is known in the art. In one embodiment, the meganuclease contains I-Seel with the recognition sequence of TAGGGATAACAGGGTAAT.

In some implementations, the IIS type restriction endonuclease recognition sequence in the cloning vector is selected from any one or any combination of the following: recognition sequences of BbsI, BsaI, BsmBI, BspQI, BsrDI, EarI, HgaI. and SfaNI.

In some implementations, the meganuclease recognition sequence in the cloning vector is selected from any one or any combination of the following: recognition sequences of I-Sea I-DmoI, f-OnuI I-PanNll, I-GzeMII, I-LtrWI and I-SmaMI.

In some embodiments, the cloning vector contains a replication origin and the selective marker gene. The selective marker gene may be selected from the antibiotic resistance gene or the ccdB gene, such as a kanamycin resistance gene, a chloramphenicol resistance gene and a neomycin resistance gene. In some implementations, the cloning vector contains a lactose operon sequence, a β galactosidase encoding gene containing the multiple cloning sites, and 3 or more BspQI recognition sequences and/or 2 or more I-sce1 recognition sequences. In some embodiments, the lactose operon sequence contains a lac promoter and a lac operator gene.

The cloning vector may be medium/high copy cloning vector. In some implementations, the cloning vector configured to construct the DNA construct of the present application is derived from: a pBR322 vector, a pUC vector, or a pET vector. In some preferred embodiments, the cloning vector configured to construct the DNA construct of the present application is derived from: the pUC vector, such as a pUC57 vector. In some implementations, the pUC vector contains a sequence as shown in SEQ ID NO:12 or is a sequence as shown in SEQ ID NO:12.

The term “derived from” used herein refers to reconstructed from, that is, the cloning vector is obtained by reconstructing an initial vector (such as the pBR322 vector, the pUC vector, or the pET vector) from which the cloning vector is derived. While the reconstruction may include: (i) the one or more IIS type restriction endonuclease and/or meganuclease recognition sequences are inserted on the initial vector such as the pBR322 vector, the pUC vector, or the pET vector; (ii) mutation is performed on the initial vector such as the pBR322 vector, the pUC vector, or the pET vector so as to generate the one or more HS type restriction endonuclease and/or meganuclease recognition sequences, or combination of (i) and (ii). In some implementations, in order to make the recognition site of the HS type restriction endonuclease on the DNA construct or the cloning vector be more and the skeleton fragment subjected to enzyme digestion be smaller, the more IIS type restriction endonuclease recognition sequences can be added on the replication origin site and an antibiotic resistance gene sequence through codon synonymous mutation.

In some implementations, the cloning vector is constructed by performing the following reconstruction on the pUC57 vector: (i) the BspQ1 recognition sequence is added after a position 1554 base and a position 2539 base of the pUC57 vector and the I-sceI recognition sequence is added after a position 1501 base and a position 2479 base; and (ii) G at a position 1397 base of the pUC57 vector is mutated into C, and AT at a position 2136 base and a position 2137 base is mutated into GC. In some implementations, the pUC vector contains a sequence as shown in SEQ NO:12 or is a sequence as shown in SEQ IL) NO:12.

In some implementations, a nucleotide sequence of the cloning vector contains a sequence as shown in SEQ) NO:1 or is a sequence as shown in SEQ ID NO: I.

DNA Construct

The present application further provides a DNA construct used in a method for producing a target DNA sequence described hereinafter. The DNA construct is autonomously replicated and contains: (a) one or more IIS type restriction endonuclease and/or meganuclease recognition sequences; (b) the target DNA sequence; and (c) protelomerase recognition sequences at lateral wings of two ends of the target DNA sequence. The DNA construct may be constructed through the following method, including: (i) providing a cloning vector containing the one or more IIS type restriction endonuclease and/or meganuclease recognition sequences; and (ii) inserting the target DNA sequence with the lateral wings at the two ends connected with the protelomerase recognition sequences into the cloning vector. In some implementations, the DNA construct is prepared by inserting the target DNA sequence with the lateral wings at the two ends connected with the protelomerase recognition sequences into multiple cloning sites of the cloning vector as mentioned above.

The “DNA construct” herein refers to a manually assembled product of a DNA fragment to be introduced into a host cell or a biosome. The DNA construct herein is autonomously replicated, that is, it may contain a sequence supporting autonomously replicating of the DNA construct in a prokaryotic or eukaryotic host cell, such as a replication origin (ori).

In some implementations, the DNA construct contains two or for example, 3, 4, 5, 6, 7, 8, 9, 10 or more endonuclease recognition sequences,

In some implementations, the DNA construct further contains a replication origin site and a selective marker gene.

In some implementations, the target DNA sequence is directly adjacent to the protelomerase recognition sequences at the two ends, that is, there is no other sequence between the target DNA sequence and the protelomerase recognition sequence at the two ends.

In some implementations, there is further other sequence between the target DNA sequence and the protelomerase recognition sequences at the two ends, such as an endonuclease recognition site and a nicking enzyme recognition site.

The two protelomerase recognition sequences at the two ends of the target DNA sequence may be subjected to direct duplication or inverted duplication.

The term “protelomerase” used herein refers to an enzyme capable of recognizing and cutting the protelomerase recognition sequences and being reconnected with a DNA containing the protelomerase recognition sequences so as to generate a closed double-stranded DNA. The protelomerase is generally found in phage, for example, but is not limited to the protelomerase coming from E. coli N15 phage (namely, protelomerase TON), Klebsiella Phi K02 phage, Yersinia Py54 phage, Halornonas Phi HAP phage, Vibrio VP882 phage, and a Borrelia burgdotfiri 1pB31.16 plasmid. In some implementations, the protelomerase is selected from the protelomerase coming from the E. coli N15 phage, the Klebsiella Phi K02 phage, the Yersinia Py54 phage, the Ifalotnonas Phi HAP phage, the Vibrio VP882 phage, and the Borrelia burgclodtlyi 1pB31.16 plasmid, or a homologue or a variant thereof. In sonic implementations, the protelomerase is the protelomerase (TelN) coming from the E. coli N15 phage, or the homologue or the variant thereof. The homologue is generally a functional homologue of the protelomerase, and its amino acid sequence may have at least 40%, 50%, 60%, 70%, 80%, 90%, 95% or 98% identity with a natural amino acid sequence of the protelomerase. The variant may include truncation, insertion, substitution and/or deficiency relative to the natural amino acid sequence of the protelomerase, for example, truncation, insertion, substitution and/or deficiency of one or more amino acids. A method for preparing and using the protelomerase is conventional, and many protelomerases can be obtained commercially.

The term “protelomerase recognition sequence” used herein is a DNA sequence capable of being recognized by the protelomerase and is determined according to the specific used protelomerase and is known in the art. In some implementations, the protelomerase recognition sequence is selected from the protelomerase recognition sequence coming from the E. coli N15 phage, the Klebsiella Phi K02 phage, the Yersinia Py54 phage, the Halomonas Phi HAP phage, the Vibrio VP882 phage, and the Borrelia burgdorferi 1pB31.16. In some implementations, the protelomerase recognition sequence comes from the E. coli N15 phage. In some implementations, the protelomerase recognition sequence contains SEQ ID NO: 2 or is composed of the same.

Method for Producing a Target DNA Sequence

The present application provides a method for producing a target DNA sequence. The term “producing” can be used interchangeably with the terms such as amplification, cloning and replication. The method includes the step of amplifying and extracting a DNA construct of the present application in a host cell, a three-step thermostatic enzyme reaction steps (namely, a first cutting reaction-a second cutting reaction-a digestion reaction) of protelomerase-IIS type restriction endonuclease and/or meganuclease-DNA exonuclease catalysis, and an optional recovery step.

In some implementations, the method for producing the target DNA sequence includes:
amplifying the DNA construct by culturing a host cell with the transferred DNA construct and extracting the amplified DNA construct from the host cell, wherein the DNA construct is autonomously replicated and contains: (a) one or more ITS type restriction endonuclease and/or meganuclease recognition sequences; (b) the target DNA sequence; and (c) protelomerase recognition sequences at lateral wings of two ends of the target DNA sequence;
enabling protelomerase to make contact with the amplified and extracted DNA construct, wherein the protelomerase recognizes and cuts the protelomerase recognition sequences on the DNA construct so as to obtain a first cutting reaction mixture;
enabling the first cutting reaction mixture to make contact with one or more IIS type restriction endonucleases and/or meganucleases, wherein the IIS type restriction endonucleases and/or meganucleases recognize and cut the IIS type restriction endonuclease and/or meganuclease recognition sequences on the construct so as to obtain a second cutting reaction mixture; and
enabling the second cutting reaction mixture to make contact with one or more exonucleases so as to digest other sequences except for the target DNA sequence.

1. Step of Amplifying and Extracting the DNA Construct

The method for producing the target DNA sequence according to the present application includes the step of amplifying and extracting the DNA construct of the present application, wherein amplification of the DNA construct is performed in the host cell.

In some implementations, the step of amplifying and extracting the DNA construct includes: amplifying the DNA construct by culturing the host cell with the transferred DNA construct and extracting the amplified DNA construct from the host cell. In some implementations, the step of amplifying and extracting the DNA construct is amplifying and extracting by utilizing the plasmid of the host cell which is publicly known in the art.

The term “host cell” used herein covers any cell capable of being converted so as to be introduced into the vector or the construct and supporting replication of the vector or the construct therein. The host cell may be a prokaryotic cell (for example, a bacterial cell, such as an E. coli cell) or a eukaryotic cell (for example, a yeast cell, an insect cell, an amphibian cell or a mammalian cell).

The DNA construct can be transferred into the host cell through any method known in the art. The method includes but is not limited to enabling the DNA construct to enter a proper competent cell, such as an E. coli competent cell, including but is not limited to a Top1.0 chemically competent cell (Invitrogen™, a product catalog number: C404010), a DH5α chemically competent cell (Invitrogen™, a product catalog number:18265017), and a DID OB chemically competent cell (Invitrogen™, a product catalog number:12331013) through chemical transformation or electroporation transformation.

Amplification of the DNA construct in the host cell is performed by culturing the host cell under a condition suitable for amplification of the DNA construct. In some embodiments, the DNA construct is fermented and cultured in a suitable liquid culture medium (for example, an LB culture medium containing a resistance gene) after the cloning vector is transfected to the host cell such as the E. coli cell. The DNA vector is also amplified in the fermented and accumulated host cell.

Extracting of the amplified DNA construct can be performed through an extracting method publicly known in the art, including but is not limited to using of an alkaline lysis method, or using of commercial plasmid extraction kit (for example, a QIAprep Spin Miniprep Kit, Qiagen).

2. First Cutting Reaction

The method for producing the target DNA sequence according to the present application further includes the step of the first cutting reaction after the above step of amplifying and extracting the DNA construct. The step of the first cutting reaction may include: enabling the protelomerase to make contact with the amplified and extracted DNA construct, wherein the protelomerase recognizes and cuts the protelomerase recognition sequences on the DNA construct so as to obtain the first cutting reaction mixture.

The first cutting reaction may be a thermostatic reaction under a temperature appropriate for protelomerase activity. The appropriate temperature is known in the art and may be for example, 20-40° C., for example, 25-35° C., and for example, 30° C. A time for the first cutting reaction may be 10 minutes to 24 hours, for example, 30 minutes to 12 hours, for example, 40 minutes, 50 minutes, 1 hour, 2 hours, or 4 hours.

In some implementations, the method for producing the target DNA sequence includes the step of inactivating the protelomerase after the first cutting reaction. The inactivating step may include: heating the protelomerase to be higher than its inactivating temperature (for example, higher than 60° C., higher than 70° C., and for example, 75° C.) and maintaining the temperature for a suitable time (for example, 2 minutes to 1 hour, for example, 5 minutes, 10 minutes, or 20 minutes).

3. Second Cutting Reaction

The method for producing the target DNA sequence according to the present application further includes the step of the second cutting reaction after the above step of the first cutting reaction. The second cutting reaction may include: enabling the first cutting reaction mixture to make contact with one or more IIS type restriction endonucleases and/or meganucleases, wherein the HS type restriction endonucleases and/or meganucleases recognize and cut the IIS type restriction endonuclease and/or meganuclease recognition sequences on the construct so as to obtain a second cutting reaction mixture.

The second cutting reaction may be a thermostatic reaction under a temperature appropriate for the used HS type restriction endonuclease and/or meganuclease. The appropriate temperature is known in the art and may be for example, 20-55° C., for example, 30-50° C., and for example, 37° C. A time for the second cutting reaction may be 10 minutes to 24 hours, for example, 30 minutes to 12 hours, for example, 40 minutes, 50 minutes, I hour, 2 hours, or 4 hours.

In some implementations, the method for producing the target DNA sequence includes the step of inactivating the IIS type restriction endonuclease and/or meganuclease after the second cutting reaction. The inactivating step may include: heating the IIS type restriction endonuclease and/or meganuclease to be higher than its inactivating temperature (for example, higher than 60° C., higher than 65° C., and for example, 65° C. or 70° C.) and maintaining the temperature for a certain time (for example, 2 minutes to 1 hour, for 1.5 example, 10 minutes, 20 minutes, or 25 minutes)

4. Digestion Reaction

The method for producing the target DNA sequence according to the present application further includes the step of the digestion reaction after the above step of the second cutting reaction. The step of the digestion reaction includes: enabling the second cutting reaction mixture to make contact with one or more exonucleases so as to digest the other sequences except for the target DNA sequence, and preferably digest all other nucleotide sequences except for the target DNA sequence.

The exonuclease may be any exonuclease known in the art, including but is not limited to phage T5 exonuclease (a phage T5 gene D15 product), phage λ exonuclease, RecE of Rac prophage, exonuclease VIII coming from E. coli, and phage T7 exonuclease (a phage T7 gene 6 product). In some implementations of the present disclosure, the exonuclease is T5 exonuclease or k exonuclease. A method for preparing and using the exonuclease is conventional, and many exonucleases can be obtained commercially.

The digestion reaction may be a thermostatic reaction under a temperature appropriate for the used exonuclease. The appropriate temperature is known in the art and may be for example, 20-55° C., for example, 30-50° C., and for example, 37° C. A time for the digestion reaction may be 10 minutes to 24 hours, for example, 30 minutes to 12 hours, for example, 1 hour, 2 hours, 3 hours, 4 hours, 5 hours or 6 hours.

In some implementations, the method for producing the target DNA sequence includes the step of inactivating the exonuclease after the digestion reaction. The inactivating step may include: heating the exonuclease to be higher than its inactivating temperature (for example, higher than 60° C., higher than 65° C., and for example, 70° C., 75° C. or 80° C.) and maintaining the temperature for a certain time (for example, 2 minutes to 1 hour, for example, 5 minutes, 10 minutes, or 20 minutes).

An additional purification step is not included after the first cutting reaction is completed and the second cutting reaction is completed. It can be understood by those skilled in the art that all the enzyme digestion reactions according to the present application 1.5 include the first cutting reaction utilizing the protelomerase and the second cutting reaction utilizing the IIS type restriction endonuclease and/or meganuclease without cutting the target DNA sequence.

In some implementations, the method for producing the target DNA sequence may further include the optional target DNA sequence recovery step after the above step of the digestion reaction. The recovery step may be performed through any one or any combinations of the following: recovering a product in the digestion step through phenol chloroform extracting and DNA adsorption centrifugal column, isopropanol/ethanol precipitation, or removing protease and salts in a reaction system through a molecular sieve chromatography supported by a high performance liquid chromatography. Endotoxin removing and/or sterile filtration treatment may further be performed when the obtained target DNA sequence is used for the mammalian cell or an animal experiment.

A product of the digestion reaction is a closed linear double-stranded DNA. In some implementations, the target DNA sequence produced by the method for producing the target DNA sequence according to the present application is the closed linear double-stranded DNA.

The method for producing the target DNA sequence according to the present application can realize high-purity (for example, the product purity is 100%) production of the target DNA sequence in a case of without any purification step. In some implementations, the method for producing the target DNA sequence does not include any step of purifying the target DNA sequence, for example, does not include the step of purifying the target DNA sequence after the digestion reaction, does not include the step of purifying the product DNA sequence after the second cutting reaction, and/or does not include the step of purifying the product DNA sequence after the first cutting reaction. In some implementations, the purity of the target DNA sequence produced by the method for producing the target DNA sequence according to the present application is greater than 95%, greater than 98%, greater than 99% or is 100%.

The method for producing the target DNA sequence according to the present application may produce the target DNA sequence on a large scale. In some implementations, a fermentation culture system of the host cell with the transferred DNA construct may be up to 1 L or above, for example, 5 L or 10 L or above. In some implementations, extracting of the amplified DNA construct may be extracting the amplified DNA construct from the fermentation culture system being 1 L or above, 5 L or above or 10 L or above. In some implementations, the produced target DNA sequence is up to 1 mg or above, 5 mg or above, or 10 mg or above.

5. Some Other Implementations

In some implementations, the DNA construct of the present application further contains an additional restriction endonuclease recognition sequence between the target DNA sequence and the protelomerase recognition sequence, and the method for producing the target DNA sequence according to the present application further includes the step of enabling the restriction endonuclease to make contact with a digestion reaction product (namely, the closed linear double-stranded DNA) so as to recognize and cut the additional restriction endonuclease recognition sequence, thus removing two terminals of the closed linear double-stranded DNA so as to prepare a double-stranded target DNA fragment with two unclosed ends (with a blunt end or a cohesive end) after the step of the digestion reaction (namely, the step of “enabling the second cutting reaction mixture to make contact with one or more exonucleases so as to digest the other sequences except for the target DNA sequence”).

In some implementations, the DNA construct of the present application further contains an additional nicking enzyme recognition sequence between the target DNA sequence and the protelomerase recognition sequence, and the method for producing the target DNA sequence according to the present application further includes the step of enabling nicking enzyme to make contact with the digestion reaction product (namely, the closed linear double-stranded DNA) so as to recognize and cut the additional nicking enzyme recognition sequence, thus removing one strand in a positive-sense strand and an antisense strand of the double-stranded DNA so as to form the DNA sequence with the two ends being of a covalently closed structure, with the local being the double-stranded DNA and with an intermediate target fragment region being single-stranded DNA after the step of the digestion reaction (namely, the step of “enabling the second cutting reaction mixture to make contact with one or more exonucleases so as to digest the other sequences except for the target DNA sequence”).

The term “nicking enzyme” used herein includes but is not limited to Nb.BbvCI, Nb.Bstra, Nb.BsrDI, Nb.BssSI, Nb.Btsi, Nt,AlwI, Nt.BbvCI, Nt.BsmAI, Nt.BspQI, Nt.BstNBI and Nt.CviPl. A method for preparing and using the nicking enzyme is conventional, and many nicking enzymes are obtained commercially (for example. New England BioLabs). The “nicking enzyme recognition sequence” is a sequence capable of being recognized and cut by the corresponding nicking enzyme, is determined according to the specific used nicking enzyme and is known in the art.

The target DNA sequence produced by the method for producing the target DNA sequence according to the present application can be imported into a cell or an animal body through a chemical or physical delivery mode to perform transient expression of an exogenous gene or integrate an exogenous DNA sequence into a genome.

In some implementations, the target DNA sequence produced by the method for producing the target DNA sequence according to the present application may include a protein coding sequence so as to be used for expression of the protein. For example, the target DNA sequence contains a promoter, a target gene and a poly(A) tail.

Therefore, the present application further provides a method for expressing a target protein. The method includes:
executing a method for producing a target DNA sequence according to the present application, wherein the target DNA sequence contains a DNA sequence for encoding the target protein;
transferring the obtained target DNA sequence into a prokaryotic cell or a eukaryotic cell; and
incubating the prokaryotic cell or the eukaryotic cell under a condition suitable for protein expression.

In some implementations, the target DNA sequence produced by the method for producing the target DNA sequence according to the present application can be used for genetic reconstruction of a target genome, for example, genetic reconstruction based on CRISPR.

Therefore, the present application further provides a method for integrating a target DNA sequence into a target integration site of a target genome. The method includes:
executing a method for producing a target DNA sequence according to the present application, wherein the target DNA sequence contains homologous arm sequences at two ends of the target integration site and an intermediate target knock-in fragment;
transferring the obtained target DNA sequence, a Cas9 protein and sgRNA designed based on the target DNA sequence together into a prokaryotic cell or a eukaryotic cell; and
incubating the prokaryotic cell or the eukaryotic cell so as to integrate the target DNA sequence into the target genome.

An optimal sgRNA sequence is designed based on the target DNA sequence through an existing sgRNA design website, for example:
https://www.genscript.com/gRNA-design-tool.html.

The homologous arm sequences at the two ends of the target integration site may be sequences about 300 bp, about 310 bp, about 320 bp, about 330 bp and about 350 by at the two ends of a target integration gene site. For example, a target integration gene is a TRAC gene or a RAB11a gene, and the homologous arm sequences at the two ends of the target integration site are sequences about 300 bp at left ends and right ends of the TRAC gene or the RAB11a gene.

The method for integrating the target DNA sequence into the target integration site of the target genome according to the present application can realize stable and continuous expression of the integrated target DNA sequence in the target genome, for example, being stably expressed for 7 days or above.

Kit

The present application further provides a kit for producing a target DNA sequence, which is configured to execute a method for producing the target DNA sequence according to the present application. The kit includes: the cloning vector, protelomerase, one or more IIS type restriction endonucleases and/or meganucleases, and one or more exonucleases. The kit may further include an operation instruction recording the method according to the present application.

The method for producing the target DNA sequence provided by the present application is suitable for industrially producing the target DNA sequence on a large scale. In some implementations, the method for producing the target DNA sequence is performed in a fermentation tank of 1 L or above, 5 L or above, 10 L or above or 20 L or above.

The method according to the present application realizes the following beneficial effects:

I. The method according to the present application is simple and universal for any sequence, does not need to specially design for each sequence, and does not need to adopt different preparation technologies for all the sequences either.

II. Because critical raw materials of the method according to the present application are the massively-extracted plasmid, and the enzyme digestion reaction in each step is performed thermostatically, scale expansion is easy.

III. The enzyme selected for the method according to the present application has very high compatibility for a reaction condition, the enzyme digestion reaction system for the next step is prepared without purifying the enzyme digestion product after each step of the enzyme digestion reaction, while the enzyme digestion system can be established in the first step of enzyme digestion. As for each step of the subsequent enzyme digestion reaction, only the enzyme reacted in the previous step needs to be inactivated, and then the new enzyme is added into the reaction system for the reaction.

IV. The method according to the present application can prepare a large amount of the high-purity target DNA sequence without any DNA stripe sorting depending on electrophoresis or DNA fragment separation based on the high performance liquid chromatography, and is very suitable for economically and efficiently preparing the gene editing template for accurate editing on a large scale and in a compliance mode.

V. The method according to the present application is easy to realize quality control particularly when being configured to industrially produce the target DNA sequence on a large scale, and is suitable for GMP production.

### BRIEF DESCRIPTION OF THE DRAWINGS

The present invention is described in more detail with reference to the following drawings:

FIG. 1 shows a functional element of a universal construct and an enzyme digestion site layout of restriction endonuclease in one embodiment of the present application.

FIG. 2 shows a production flow f a target DNA sequence of one implementation of the present application.

FIG. 3 shows a result of detecting an intermediate product and an end product of double-stranded DNA through agarose gel electrophoresis in one embodiment of the present application. A lane M is a 3000 by double-stranded DNA marker, a lane 1 is a product of a purified preparation vector subjected to TelN enzyme digestion, a lane 2 is a product of the lane 1 product subjected to I-sceI enzyme digestion, and a lane 3 is a product of the lane 2 product digested by λ exonuclease.

FIG. 4 shows purity verifying of an end product target sequence 1 through Agilent Bioanalyzer 2100 in one embodiment of the present application.

FIG. 5 shows a result of detecting an intermediate product and an end product of double-stranded DNA through agarose gel electrophoresis in one embodiment of the present application. A lane M is a 3000 bp double-stranded. DNA marker, a lane 1 is a product of a purified preparation vector subjected to TelN enzyme digestion, a lane 2 is a product of the lane 1 product subjected to BspQI enzyme digestion, and a lane 3 is a product of the lane 2 product digested by k exonuclease.

FIG. 6 shows purity verifying of an end product target sequence 2 through Agilent Bioanalyzer 2100 in one embodiment of the present application.

FIG. 7 shows detection of cell viability after electroporation of a target sequence 2 prepared according to a method of the present application in one embodiment of the present application.

FIG. 8 shows detection of green fluorescent protein expression of a cell 48 hours after electroporation of a target sequence 2 prepared according to a method of the present application in one embodiment of the present application.

FIG. 9 shows detection of cell viability after electroporation of a target sequence 2 prepared according to a method of the present application in one embodiment of the present application.

FIG. 10 shows detection of green fluorescent protein expression of a cell 7 days after electroporation of a target sequence 2 prepared according to a method of the present application in one embodiment of the present application.

### DETAILED DESCRIPTION

Unless otherwise specified, the technical and scientific terms used in the present invention have the meanings commonly understood by those skilled in the art to which the present invention belongs.

The technical solution in this disclosure is further described in detail with reference to the accompanying drawings and embodiments. Unless otherwise specified, the methods and materials in the examples described below are commercially available and conventional products. Those skilled in the art to which the present disclosure belongs will understand that the method and materials described below are only exemplary, and should not be regarded as limiting the scope of the present disclosure.

### EXAMPLE 1: Design and Construction of a Universal Construct

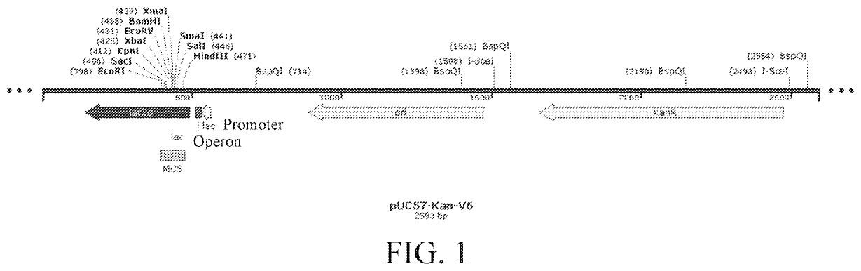

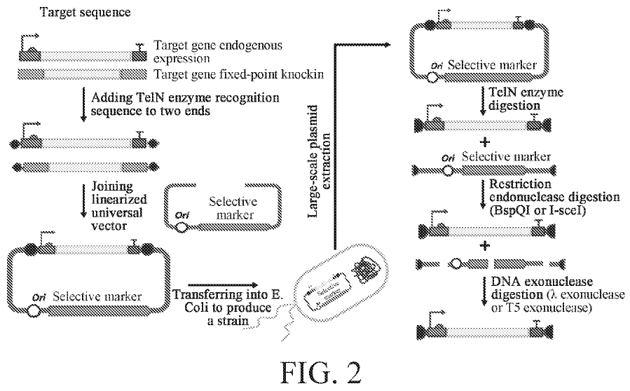

A pUC57 kanamycin resistance vector is selected as a reconstructing vector. As shown in FIG. 1, on the basis that only a BspQI enzyme digestion recognition site originally exists at a position 714 base, the two BspQI enzyme digestion recognition sites (sequence: GCTCTTC) are added after a position 1554 base and position 2539 base between functional elements respectively. Two meganuclease I-sceI. enzyme digestion recognition sequences (sequence: TAGGGATAACAGGGTAAT) not existing originally are knocked in after a position 1501 A base and a position 2479 A base, In addition. G is also changed into C at a position 1397 base through point mutation so as to realize codon synonymous mutation, and in this way, one BspQi enzyme digestion recognition site is added on its encoding gene under a situation of ensuring ori being unchanged in function. Similarly, through synonymous mutation (AT to GC), the base AT is mutated into GC at position 2136 and position 2137, and one BspQI enzyme digestion recognition site is added in the encoding gene of a kanamycin resistance gene. The above-mentioned sequence insertion and point mutation are both subjected to site-directed mutation service through Nanjing GenScript Biotech Corp. on the basis of a pUC57-KanR plasmid vector (Nanjing GenScript Bioteth Corp., sequence information please see: https://www.addgene.orglvector-database/6258/, SEQ ID NO:12), and a universal preparation construct as shown in FIG. 1 is obtained. The functional element of the universal construct includes from left to right: an existing lactose operon sequence for blue-white spot screening of the pUC57-KanR vector, including a lac promoter, a lac operator gene, and a β galactosidase encoding gene lacZa containing a multiple cloning site (MSC); a plasmid replication origin site sequence ori, the kanamycin resistance gene KanR, and the added BspQI recognition sequence GCTCTTC and the I-sceI enzyme digestion recognition sequence TAGGGATAACAGGGIAAT. The designed sequence of the universal construct is as shown in SEQ ID NO:l.

### EXAMPLE 2: Target Sequence 1 Configured to Knock GH) Gene into RABIla Gene

2.1 Design of Target Sequence 1

The knocked-in sequences at two ends of a GFP sequence site are selected from sequences (as RAB11a homologous arm sequences) of 300 bp on the left and right of a human genome RAB11a (Ras-related protein Rab-11A) gene knock-in site respectively, as shown in SEQ ID NO:4 and SEQ If) NO:5 respectively. An original sequence of the designed target sequence 1 (1356 bp stable double-stranded DNA) is as shown in SEQ ID NO:3, and protelomerase TeIN recognition sequences TATCAGCAC.ACAATTGCCCATTAT.ACGCGCGTATAATGGACTATTGTGTGCTGA TA (SEQ If) NO:2) coming from E. coli N15 phage are added to the two ends of the target DNA sequence 1. The sequence of the target sequence 1 after the two ends are added with the protelornerase TelN enzyme recognition sequences is as shown in SEQ ID NO:6. A designed end sequence is subjected to complete-sequence gene synthesis by the Nanjing GenScript Biotech Corp. (https://www.genscript.com.cn/gene synthesis,html).

2.2 DNA Construct Preparation and Large-Scale Plasmidplasrrrrd Preparation

The target sequence 1 (SEQ ID NO:6) synthesized in step 2.1 and with the two ends added with the TelN enzyme recognition sequences is flat joined into an enzyme digestion site of the universal vector plL1C57-Kan-V6 (the universal vector prepared in embodiment 1) subjected to single enzyme digestion linearization through restriction endonuclease EcoRV (New England BioLabs, Catalog. #R3195L) by T4 ligase (Thermo Scientific™, product catalog number: EL0011). A joining product is converted to an E. coli competent cell through an electroporation method, the transformed E. coli is coated on an LB plate culture medium with kanamycin and placed at a temperature of 37° C. for overnight culture. 10 single colonies are picked on the next day to be subjected to liquid culture on 3 mL of LB liquid culture mediums containing 50 μg/ml kanamycin and subjected to plasmid extraction. Each plasmid is identified through a Sanger sequencing means, and the plasmids with the sequences being entirely correct are determined. Plasmid extraction and sequencing are completed by Nanjing GenScript Biotech Corp. (https://www.genscript.com.cnlcustom-plasmid-preparation.html). Picked E. coli strains containing the plasmids with the correct sequences are streaked and preserved. The preserved E. coli strains are inoculated to prepare a seed solution (an OD value is about 0,8), and then the seed solution is 1% inoculated to 1 L of an E. coli culture system so as to perform 10 L of large-scale plasmid extraction.

2.3 Preparation of Stable Double-Stranded DNA Containing Target Sequence 1

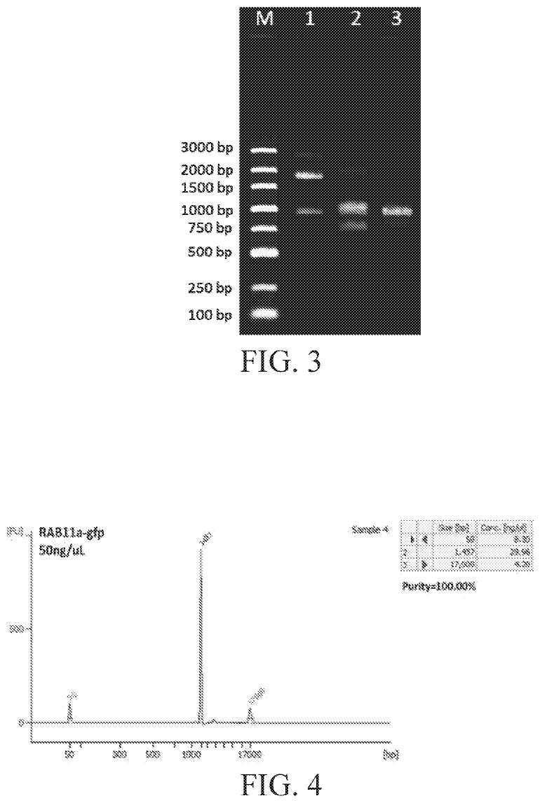

Plasmid preparation vectors subjected to large-scale extraction are subjected to three-step thermostatic enzyme digestion reaction so as to obtain the linear closed double-stranded DNA containing the target sequence 1. Step I, the 0.8 mg annular preparation vectors are cut into two double-stranded DNA linear fragments with the two ends closed and respectively containing the target sequence 1 and vector skeletons through TeIN enzyme (New England BioLabs, Catalog #M0651S, and 1 id, 5 of enzyme is added into each 300 fmol of TeIN recognition sites), incubation is performed for 1 hour at 30° C., then heating is performed for 10 minutes at 75° C. so as to inactivate the TeIN enzyme. After the reaction is completed, whether the reaction is completely performed or not is detected through an agarose electrophoresis method, that is whether the preparation vectors are completely changed from the superspiral annular plasmids into the two linearized double-stranded DNA, and stripe positions should be target fragment and preparation vector skeleton sizes (about 2.6 kb) respectively. Step II, the target sequence 1 contains the BspQJ recognition sequence GCTCITC, an 1-sceI enzyme with the target sequence 1 not containing the I-sceI enzyme recognition sequence is adopted, the target sequence 1 is made to remain unchanged, while the vector skeleton sequences will be cut into a plurality of broken DNA fragments containing double strands through the I-sceI enzyme (New England BioLabs, Catalog #R0694L). The reaction condition is that the reaction is performed for 1 hour at 37° C. and then inactivation is performed. After the reaction is completed, whether the reaction is completely performed or not is detected through the agarose electrophoresis method. Step HI, the shredded vector skeleton DNA fragments are completely digested through DNA exonuclease. After the reaction in Step II, the exonuclease, namely, exonuclease (New England. BioLabs, Catalog #M0262L) or T5 exonuclease (New England. BioLabs, Catalog #1\40663L) is added into the reaction system to be reacted for 2 hours at 37° C., and then is subjected to thermal inactivation, The specific reaction condition please see the following Table 1 to Table 3. After the reaction, the purity of the target product is detected through agarose gel electrophoresis, and the product is a single product (see FIG. 3) only having a target stripe. The end product, namely the stable double-stranded target sequence 1 is subjected to purity verifying through Agilent Bioanalyzer 2100, The DNA pure product (see FIG. 4) with the 100% purity and only containing the target fragment can be obtained without any additional DNA molecular fragment sorting or purification step.

TABLE 1
TelN enzyme digestion reaction system and reaction condition

| Component | RAB11a-gfp mL |
|---|---|
| 10 × ThermoPol reaction solution | 2 |
| Plasmid DNA (0.8 mg) | 1.13 |
| TelN protelomerase (50 U/μL) | 0.23 |
| ddH2O | 6.64 |
| Total volume | 10 |

Reaction condition:
30° C., 1 h
75° C., 5 min
4° C. till the next step

TABLE 2
I-sceI enzyme digestion reaction system and reaction condition

| Component | RAB11a-gfp mL |
|---|---|
| Above-mentioned TelN enzyme digestion product | 10.00 |
| I-sceI (50 U/μL) | 0.40 |
| ddH2O | 0.00 |

Reaction condition:
37° C., 60 min
65° C., 20 min
4° C. till the next step

TABLE 3
DNA exonuclease reaction system and reaction condition

| Component | RAB11a-gfp mL |
|---|---|
| Above-mentioned I-sceI enzyme digestion product | 10.400 |
| Lambda exonuclease | 0.06 |
| ddH2O | 0.00 |

Reaction condition:
37° C., 3 h
Adding 0.4 mL 0.25M EDTA till an end concentration is 10 mM
75° C., 10 min
4° C. till the next step

As shown in FIG. 3, a lane M is a 3000 by double-stranded DNA marker. A lane 1 is a product of the purified preparation vector subjected to TelN enzyme digestion, wherein the product includes a 1 kb target stripe double-stranded DNA and a 2 kb vector skeleton double-stranded DNA. A lane 2 is a product after the lane 1 product is subjected to I-sceI enzyme digestion, wherein the product includes a 1 kb target stripe double-stranded DNA and a vector skeleton double-stranded DNA cut into two fragments. A lane 3 is a product after the lane 2 product is digested by Lambda Exo exonuclease, and the end product only has the 1 kb target stripe double-stranded DNA.

As shown in FIG. 4, the end product is subjected to purity detection through Agilent Bioanalyzer capillary electrophoresis and a DNA 12000 detection chip. A 50 bp peak and a 17,000 bp peak are internal reference peaks of chip detection, a material peak of the target sequence only exists in the overall detection range, and the purity is 100%.

2.4 Final Purification of Stable Double-Stranded DNA Containing Target Sequence 1

The above-mentioned reaction product may be recovered through phenol chloroform extracting (Invitrogen™, product catalog number: 15593031), and a DNA adsorption centrifugal column (QIAGEN-tip 100, Qiagen, a product catalog number: 10043) made of a special material, and then DNA molecules only containing the target sequence are recovered through isopropanol (Sinopharm Chemical Reagent Co., Ltd., serial number 80109218)/ethanol (Sinopharm Chemical Reagent Corporation, Ser. No. 10009257) precipitation. The isopropanol:/ethanol precipitation method adopted in the embodiment includes the specific steps: 1) 0.7-time volume of normal-temperature isopropanol (for example, 0.7 ml of isopropanol is added into 1 ml of to-be-concentrated stable double-stranded DNA) is added to be mixed uniformly, then centrifugation is performed for 10 minutes at 4° C. at 12,000-14,000 rpm, and a supernatant is absorbed carefully to avoid touch of the precipitation; 2) 1 ml of normal-temperature 70% ethanol solution is added, the plasmid precipitation is gently suspended for adequate washing, centrifugation is performed for 5-10 minutes at 4° C. at 12,000-14,000 rpm, and a supernatant is absorbed carefully to avoid touch of the precipitation; 3) centrifugation is performed for 5-10 seconds at 4° C. at 5,000-10,000 rpm, and residual liquid is absorbed completely and carefully through a 20-microliter or 200-microliter pipettor to avoid touch of the precipitation; and 4) after no obvious liquid (drying may be finished generally in 1 minute after the liquid is absorbed completely) is observed by naked eyes, a solution with an appropriate volume (for example, solution V, 10 mM Tris-C1 pH8.5 or Milli-Q-level pure water) is added to dissolve DNA.

The 0.8 mg of the purified preparation vectors containing the target sequences are adopted in the embodiment, wherein a length of the target sequences accounts for 35% of the overall preparation vectors, that is, the theoretical stable double-stranded DNA product should be 0.28 mg. After isopropanol precipitation and purification, the stable double-stranded DNA only containing the target sequence of the obtained pure product is 0.18 mg through O.D. 260 ultraviolet absorption measurement (Nanodrop One, ThermoFisher), and productivity is 64.73%. By amplifying the reaction system, the 4.5 mg of the purified preparation vectors containing the target sequences are put at a time so as to ensure that the 1 mg of end product is obtained at a time. The 1 mg or above of the plasmid is purified through a molecular sieve chromatography (a chromatographic column filler: Sepharose 6 Fast Flow, Cytiva) supported by a high performance liquid chromatography (AKTA. Explorer 100, Cytiva) so as to remove protease and salts in the reaction system.

### EXAMPLE 3: Target Sequence 2 Configured to Knock Gene Editing Template of GFP with CMV Promoter into TRAC Gene

3.1 Design of Target Sequence 2

Knocked-in sequences at two ends of a GFP sequence site and with the CMV promoters are selected from sequences (as TRAC left and right homologous arm sequences) of 300 bp on the left and right of a human genome TRA.0 (T cell receptor a chain encoding gene) gene knock-in site respectively, as shown in SEQ ID NO:7 and SEQ ID NO:8 respectively. An original sequence of the target sequence 2 (1885 bp stable double-stranded DNA) is as shown in SEQ ID—N0:9, and protelomerase TelN recognition sequences TATCAGCACACAATTGCCCATTATACGCGCGTATAATGGACTATTGTGTGCTGA TA (SEQ ID NO:2) coming from E. coli N15 phage are added to the two ends of the target DNA sequence 2. The sequence of the target sequence 2 after the two ends are added with the protelomerase TelN enzyme recognition sequences is as shown in SEQ ID NO:10, A designed end sequence is subjected to complete-sequence gene synthesis by the Nanjing GenScript Biotech Corp. (haps://www.genscript.com.cn/gene_synthesis.html).

3.2 DNA Construct Preparation and Large-Scale Plasmid Preparation

Same as the embodiment 2, the target sequence 2 synthesized in 3.1 and with the two ends added with the TelN enzyme recognition sequences is flat joined into pliC57-Kan-V6 (the universal vector prepared in embodiment 1) subjected to single enzyme digestion linearization through restriction endonuclease EcoRV (New England BioLabs, Catalog R3195L) by T4 ligase (Thermo Scientific™ product catalog number: EL0011). A. joining product is converted to an E. coli competent cell, the transformed E. coli is coated on an LB plate culture medium with kanamycin and placed at a temperature of 37° C. for overnight culture. 10 single colonies are picked on the next day to be subjected to liquid culture on 3 mL of LB liquid culture mediums containing 50 μg/ml kanamycin and subjected to plasmid. extraction. Plasmid extraction is completed by Nanjing GenScript Biotech Corp. (https://www.genscript.com.cn/custom-plasmid-preparation.html). Each plasmid is identified through a sequencing means, and the plasmids with the sequences being entirely correct are determined. E. coli strains containing the plasmids with the correct sequences are streaked and preserved. 10 L of large-scale plasmid extraction is performed by preparing a seed solution and inoculating the seed solution to 1 L of an E. coli culture system.

3.3 Preparation of Stable Double-Stranded DNA Containing Target Sequence 2

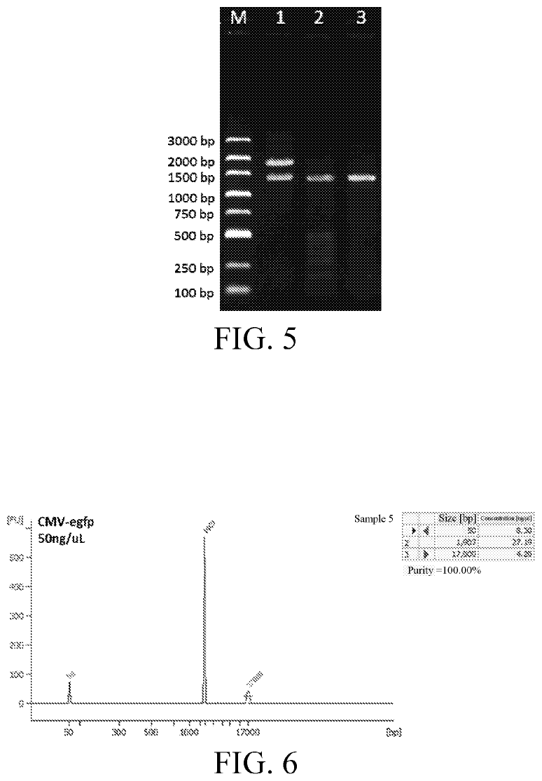

Same as operation steps in embodiment 2,3, plasmid preparation vectors subjected to large-scale extraction are subjected to three-step thermostatic enzyme digestion reaction so as to obtain the closed double-stranded DNA containing the target sequence. Step I, the annular preparation vectors obtained in embodiment 3.2 are cut into two double-stranded DNA linear fragments with the two ends closed and respectively containing the target sequence 2 and vector skeletons through TelN enzyme (New England BioLabs, Catalog #M0651S, and 1 μL 5 U/μL of enzyme is added into each 300 fmol of TelN recognition sites), and the specific reaction conditions see Table 4. After the reaction is completed, whether the reaction is completely performed or not is detected through the agarose electrophoresis method. Step II, the target sequence 2 may be subjected to enzyme digestion through I-sce used in embodiment I. However, the target sequence does not contain the BspQI recognition sequence, BspQI with more enzyme digestion sites on the universal vector may be adopted for enzyme digestion, the target fragment is made to remain unchanged, while the vector skeleton sequences will be cut into a plurality of broken DNA fragments containing double strands, and the specific reaction conditions see Table 5. After the reaction is completed, whether the reaction is completely performed or not is detected through the agarose electrophoresis method. Step III, the shredded vector skeleton DNA fragments are completely digested through DNA exonuclease. After the reaction in Step H, the 2, exonuclease (New England BioLabs, Catalog #M0262L) or T5 exonuclease (New England BioLabs, Catalog #M0663L) are added into the reaction system to be reacted for 2 hours at 37° C., and then is subjected to thermal inactivation. The specific reaction conditions see Table 6. The purity of the target product is detected through agarose gel electrophoresis, and the product is a single product (see FIG. 5) only having a target stripe. The end product, namely the stable double-stranded target sequence 2 is subjected to purity verifying through Agilent Bioanalyzer 2100, As shown in FIG. 6, a 50 bp peak and a 17,000 bp peak are internal reference peaks of chip detection, a material peak of the target sequence only exists in the overall detection range, and the purity is 100%. The DNA pure product with the 100% purity and only containing the target fragment can be obtained without any additional DNA molecular fragment sorting or purification step.

TABLE 4
TelN enzyme digestion reaction system and reaction condition

| Component | CMV-eGFP mL |
|---|---|
| 10 × ThermoPol reaction solution | 1.2 |
| Plasmid DNA (1.2 mg) | 1.57 |
| TelN protelomerase (50 U/μL) | 0.31 |
| ddH2O | 8.92 |
| Total volume | 12 |

Reaction condition:
30° C., 1 h
75° C., 5 min
4° C. till the next step

TABLE 5
BspQI enzyme digestion reaction system and reaction condition

| Component | CMV-eGFP mL |
|---|---|
| TelN enzyme digestion product | 12.00 |
| BspQI (4000 U/μL) | 0.0225 |
| ddH2O | 0.00 |

Reaction condition:
50° C., 60 min
80° C., 20 min
4° C. till the next step

TABLE 6
DNA exonuclease reaction system and reaction condition

| Component | CMV-eGFP mL |
|---|---|
| BspQI enzyme digestion product | 12.023 |
| Lambda Exo exonuclease | 0.10 |
| ddH2O | 0.00 |

Reaction condition:
37° C., 3 h
Adding 0.48 mL 0.25M EDTA till an end concentration is 10 mM
75° C., 10 min
4° C. till the next step

As shown in FIG. 5, a lane M is a 3000 bp double-stranded DNA marker. A lane 1 is a product of the purified preparation vector subjected to TeIN enzyme digestion, wherein the product includes a 1.8 kb target stripe double-stranded DNA and a 2 kb vector skeleton double-stranded DNA. A lane 2 is a product after the lane 1 product is subjected to BspQI enzyme digestion, wherein the product includes a 1.8 kb target stripe double-stranded DNA and a vector skeleton double-stranded DNA cut into five fragments. A lane 3 is a product after the lane 2 product is digested by 2, exonuclease, and the end product only has the 1.8 kb target stripe double-stranded DNA.

As shown in FIG. 6, the end product is subjected to purity detection through Agilent Bioanalyzer capillary electrophoresis and a DNA 12000 detection chip. A 50 bp peak and a 17,000 bp peak are internal reference peaks of chip detection, a material peak of the target sequence only exists in the overall detection range, and the purity is 100%.

3.4 Final Purification of Stable Double-Stranded DNA Containing Target Sequence 2

The above-mentioned reaction product may be recovered through phenol chloroform extracting (Invitrogen™, product catalog number: 15593031), and a DNA adsorption centrifugal column (QIAGEN-tip 100, Qiagen, a product catalog number: 10043) made of a special material, and then isopropanol (Sinopharm Chemical Reagent Co., Ltd., serial number 80109218)/ethanol (Sinopharm Chemical Reagent Corporation, Ser. No. 100092:57) precipitation is conducted. The isopropanol/ethanol precipitation method adopted in the embodiment includes the specific steps: 1) 0.7-time volume of normal-temperature isopropanol (for example, 0.7 ml of isopropanol is added into 1 ml of to-be-concentrated stable double-stranded DNA) is added to be mixed uniformly, then centrifugation is performed for 10 minutes at 4° C. at 12,000-14,000 rpm, and a supernatant is absorbed carefully to avoid touch of the precipitation; 2) 1. ml of normal-temperature 70% ethanol solution is added, the plasmid precipitation is gently suspended for adequate 1.5 washing, centrifugation is performed for 5-10 minutes at 4° C. at 12,000-14,000 rpm, and a supernatant is absorbed carefully to avoid touch of the precipitation; 3) centrifugation is performed for 5-10 seconds at 4° C. at 5,000-10,000 rpm, and residual liquid is absorbed completely and carefully through a 20-microliter or 200-microliter pipettor to avoid touch of the precipitation; and 4) after no obvious liquid (drying may be finished generally in 1 minute after the liquid is absorbed completely) is observed by naked eyes, a solution with an appropriate volume (for example, solution V, 10 mM Tris-CI pH8.5 or Milli-Q-level pure water) is added to dissolve DNA.

The 1.2 mg of the purified preparation vectors containing the target sequences are adopted in the embodiment, wherein a length of the target sequences accounts for 42.5% of the overall preparation vectors, that is, the theoretical stable double-stranded DNA product should be 0.51 mg. After isopropanol preparation and purification, the stable double-stranded DNA only containing the target sequence of the obtained pure product is 026 mg through O.D. 260 ultraviolet absorption measurement (Nanodrop One, ThermoFisher), and productivity is 50.98%. By amplifying the reaction system, the 4.62 mg of the purified preparation vectors containing the target sequences are put at a time so as to ensure that the 1 mg of end product is obtained at a time. The 1 mg or above of the plasmid is purified through a molecular sieve chromatography (a chromatographic column filler: Sepharose 6 Fast Flow, Cytiva) supported by a high performance liquid. chromatography (AKTA Explorer 100, Cytiva) so as to remove protease and salts in the reaction system.

### EXAMPLE 4: Electroporation of Target Sequence 2 Prepared in Example 3 into HEIX1293T Cell Line for Expression of Green Fluorescent Protein

4.1 Culture and preparation of HEK293T mammalian cell line: a culture medium (DMEM Low-glucose, Gibco™, Catalog number:11885084) is taken out of a 4° C. refrigerator, and placed at a super clean bench at the room temperature. A HEK293T cell (ATCC® CRL-3216™) freezing tube is held by the hand to be shaken at 37° C. to be thawed. 1 mL of cell freezing medium is added into 5 mL of culture medium to remove DMSO, centrifugation is performed, and then a supernatant is abandoned, 5 mL of fresh culture medium is added, culturing is performed in a 6 cm plate, and 1*10/\6 cells/bottle are added to a 10 cm culture dish to be cultured for 48 h.

4.2 Electroporation of DNA to HEK293T cell line

1. Cell counting is 1.96*10∧6/mL and the total is 5 mL.

2. 10 μL of electroporation liquid+1 μg/μL of target sequence 2 encoding a green fluorescent protein gene or 2 μL of a plasmid sample extracted in embodiment 3.2

3. 1 mL of the cells obtained in step 1 are taken to be centrifuged at low speed, supernatant is abandoned, and then the 10 μL of electroporation liquid is added for resuspension.

4. The target sequence 2 or the plasmid sample in step 2 and the cells in step 3 are incubated for 10 min at the room temperature.

5. The sample and cells incubated in step 4 are mixed uniformly, and then incubated for 10 min at the room temperature.

6. A Celetrix CTX-1500 LE electroporator is adopted for electroporation with the voltage of 420 V, and three groups are tested for each sample.

7. The cells are cultured in the culture dish.

8. After the cells are cultured for 48 h, a flow cytometer (CytoFLEX, BECKMAN COULTER) is used for observing a proportion of living cells and a proportion of cell population with green fluorescent protein expression.

4.3 Test Result

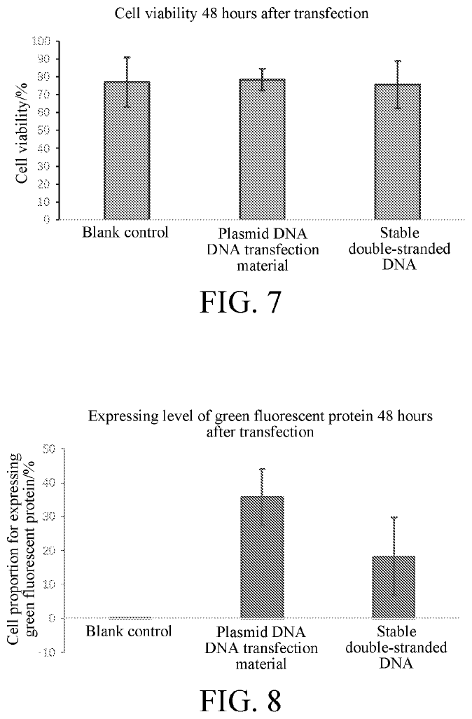

As shown in FIG. 7, viability detection is counted and determined by selecting the cell populations with the normal forms through the flow cytometer, and viability for the cell sample with electroporation of 2 μg of the stable double-stranded DNA target sequence 2 is close to that for electroporation of 2 μg of plasmid and that of a blank control group without electroporation of any DNA.

As shown in FIG. 8, the green fluorescent protein express level detection selects the cells expressed positive in the green fluorescent protein in the identified living cell population through the flow cytometer and counts a percentage. A GFP expression rate average value for the cell sample with electroporation of 2 mg of the stable double-stranded DNA is 18.21%, a GFP expression rate average value for electroporation of 2 μg of the plasmid is 35.70%, while a GFP expression rate average value for the blank control group without electroporation of any DNA is 0.36%. It indicates that the transferred stable double-stranded DNA can be expressed in the cells. A GFP expression rate of the HEK293 cell with electroporation of 2 μg of the plasmid is two times that of the cell with electroporation of 2 μg of the stable double-stranded DNA, and this is possibly because that the plasmid is easier to enter cytoplasm to be subjected to transient transfection expression due to its superhelical structure.

EXAMPLE 5: Electroporation of Target Sequence 2 Prepared in Example 3 and CRISPR-Cas9 RNP Compound Together into HEK293T Cell Line for Genome Fixed-Point Knock-In of GFP Expressed Gene

5.1 Culture and preparation of HEK293T mammalian cell line: HEK293T cells are revived through the method same as that of embodiment 4.1.

5.2 Electroporation of DNA to HEK293T cell line 1. Cell counting is 1.96*10A6/mL, and the total is 5 mL.

2. 10 μL of electroporation liquid+1 μg/μL of stable double-stranded DNA target sequence 2 encoding a green fluorescent protein gene or 2 μL of a plasmid sample extracted in embodiment 3.2; and 0.5 μL of 50 pmol Cas9 protein (GenCrispr Cas9-N-NLS Nuclease, GenScript, Cat. No. 203388)+1 μL. of 200 pmol sgRNA (the sequence: AGAGUCUCUCAGCUGGUACAguuuuagagcuaGAAAuagcaaguuaaaauaaggcuaguccguua ucaacuugaaaaaguggcaccgagucggugcuuuu, SEQ ID NO:11, after being designed in a design website https://www.genscriptcom/gRNA-design-tool,html based on the target DNA sequence, it is synthesized by Nanjing Genscript) (gene knock-in experimental group) are added to be incubated for 10 min at the room temperature, and that without adding Cas9 protein and sgRNA is taken as the blank control group.

3. 1 mL of the cells obtained in step 1 are taken to be centrifuged at low speed, supernatant is abandoned, then the 10 μL of electroporation liquid is added for resuspension, and then incubation is performed for 10 min at the room temperature.

4. 2 and 3 are mixed uniformly, then incubation is performed for 10 min at the room temperature, and then a mixture is transferred into an electroporation cup.

5. A Celetrix CTX-1500 LE electroporator is adopted for electroporation with the voltage of 420 V, and three groups are tested for each sample.

6. The cells are cultured in the culture dish.

7. After the cells are cultured for 7 days, a flow cytometer is used for observing a proportion of living cells and a proportion of cell population with green fluorescent protein expression.

5.3 Test Result

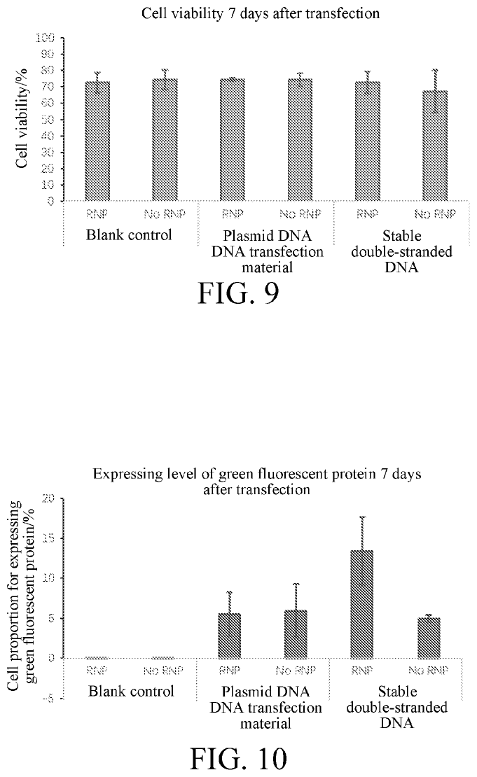

As shown in FIG. 9, viability detection is counted and determined by selecting the cell populations with the normal forms through the flow cytometer, and whether adding the Cas9 protein and sgRNA compound (RNP, ribonucleoprotein) or not, viability for the cell sample with electroporation of 2 μg of the stable double-stranded DNA is close to that for electroporation of 2 μg of plasmid and that of a blank control group without electroporation of any DNA.

The green fluorescent protein express level detection selects the cells expressed positive in the green fluorescent protein in the identified living cell population through the flow cytometer and counts a percentage. As shown in FIG. 10, whether adding the Cas9 protein and sgRNA compound (RNP) or not, a GFP expression rate average value for the blank control group without electroporation of any DNA is roughly equal to 0. GFP expression rate average values for electroporation of 2 μg of the plasmid are very close and are 5.51% and 5,91% respectively, and it shows that an experimental group with the added Cas9 and sgRNA RNP compound is not massively subjected to knock-in of the GFP gene fragments on the HEK293T cell genome and subjected to GFP background expression caused by plasmid template residual. GFP expression rate average values for the cell sample with electroporation of 2 μg of the stable double-stranded DNA are 13.35% and 4.92% respectively. Compared with the control group without RNP, GFP expression of the stable double-stranded DNA 7 days after being subjected to electroporation into the FIEK293T cell line is still 13.35%. It shows that knock-in of the GFP encoding gene based on gene editing occurs, and the stable double-stranded DNA is more beneficial to fixed-point knock-in based on CRISPR mediation and has lower template residual than that of the plasmid. (An expression background of the green fluorescent protein is slightly lower than that of the plasmid, but fixed-point knock-in based on CRISPR mediation can be significantly improved.)

The implementations of the present invention are not limited to those described in the above embodiments. Without departing from the spirit and scope of the present invention, those of ordinary skill in the art can make various modifications and improvements to the present invention in form and details, and these are deemed to fall within the protection scope of the present invention.

1. A method for producing a target DNA sequence, comprising:
a step of amplifying and extracting a DNA construct, comprising: amplifying the DNA construct by culturing a host cell with the transferred DNA construct and extracting the amplified DNA construct from the host cell, wherein the DNA construct is autonomously replicated and contains: (a) one or more IIS type restriction endonuclease and/or meganuclease recognition sequences; (b) the target DNA sequence; and (c) protelomerase recognition sequences at lateral wings of two ends of the target DNA sequence;
a step of a first cutting reaction, comprising: enabling protelomerase to make contact with the amplified and extracted DNA construct, wherein the protelomerase recognizes and cuts the protelomerase recognition sequences on the DNA construct so as to obtain a first cutting reaction mixture;
a step of a second cutting reaction, comprising: enabling the first cutting reaction mixture to make contact with one or more IIS type restriction endonucleases and/or meganucleases, wherein the IIS type restriction endonucleases and/or meganucleases recognize and cut the IIS type restriction endonuclease and/or meganuclease recognition sequences on the construct so as to obtain a second cutting reaction mixture; and
a step of a digestion reaction, comprising: enabling the second cutting reaction mixture to make contact with one or more exonucleases so as to digest other sequences except for the target DNA sequence.

2. The method according to claim 1, wherein the protelomerase is selected from the protelomerase coming from E. coli N15 phage, Klebsiella Phi K02 phage, Yersinia Py54 phage, Halomonas Phi HAP phage, Vibrio VP882 phage, and a Borrelia burgdorferi 1pB31.16 plasmid, or a homologue or a variant thereof.

3. The method according to claim 1, wherein the DNA construct contains two or more, for example, 3, 4, 5, 6, 7, 8, 9, 10 or more IIS type restriction endonuclease and/or meganuclease recognition sequences, wherein the IIS type restriction endonuclease is selected from one or any combination of the following: BbsI, BsaI, BsmBI, BspQI, BsrDI, EarI, HgaI and SfaNk, wherein the meganuclease is selected from any one or any combination of the following: I-Seel, I-CreI, I-DmoI, I-OnuI, I-LtrI, I-PanMI, I-GzeMII, I-HjeI, I-LtrWI and I-SmaMI.

4-5. (canceled)

6. The method according to claim 1, wherein the exonuclease is T5 exonuclease or λ exonuclease.

7. The method according to claim 1, wherein the DNA construct contains a replication origin and a selective marker gene.

8. The method according to claim 1, wherein an additional purification step is not comprised after the first cutting reaction is completed and the second cutting reaction is completed.

9. The method according to claim 1, wherein the step of the first cutting reaction is thermostatically performed under a temperature appropriate for protelomerase activity, the step of the second cutting reaction is thermostatically under a temperature appropriate for the IIS type restriction endonuclease and/or meganuclease, and/or the step of the digestion reaction is thermostatically performed under a temperature appropriate for the exonuclease.

10. The method according to claim 1, further comprising a step of inactivating the protelomerase after the first cutting reaction, inactivating the IIS type restriction endonuclease and/or meganuclease after the second cutting reaction, and/or inactivating the exonuclease after the digestion reaction.

11. The method according to claim 1, wherein the DNA construct is constructed through the following method, comprising: (i) providing a cloning vector containing the one or more IIS type restriction endonuclease and/or meganuclease recognition sequences; and (ii) inserting the target DNA sequence with the lateral wings at the two ends connected with the protelomerase recognition sequences into the cloning vector.

12. The method according to claim 11, wherein the cloning vector is a plasmid, wherein the cloning vector is derived from: a pBR322 vector, a pUC vector, or a pET vector.

13. (canceled)

14. The method according to claim 1, wherein the host cell is an E. coli cell.

15. The method according to claim 1, wherein the DNA construct further contains an additional restriction endonuclease recognition sequence between the target DNA sequence and the protelomerase recognition sequence, and the method further comprises a following step: enabling the restriction endonuclease to make contact with a digestion reaction product so as to recognize and cut the additional restriction endonuclease recognition sequence, so as to prepare a double-stranded target DNA fragment with a blunt end or a cohesive end after the step of the digestion reaction.

16. The method according to claim 1, wherein the DNA construct further contains an additional nicking enzyme recognition sequence between the target DNA sequence and the protelomerase recognition sequence, and the method further comprises a following step: enabling nicking enzyme to make contact with a digestion reaction product so as to recognize and cut the additional nicking enzyme recognition sequence, so as to prepare a DNA sequence with two ends being end-closed double-stranded DNA and with an intermediate being single-stranded DNA after the step of the digestion reaction.

17. A method for expressing a target protein, comprising:
executing the method for producing the target DNA sequence according to claim 1, wherein the target DNA sequence contains a DNA sequence for encoding the target protein;
transferring the obtained target DNA sequence into a prokaryotic cell or a eukaryotic cell; and
incubating the prokaryotic cell or the eukaryotic cell under a condition suitable for protein expression.

18. A method for integrating a target DNA sequence into a target integration site of a target genome, comprising:
executing the method for producing the target DNA sequence according to claim 1, wherein the target DNA sequence contains homologous arm sequences at two ends of the target integration site and an intermediate target knock-in fragment;
transferring the obtained target DNA sequence, a Cas9 protein and sgRNA designed based on the target DNA sequence together into a prokaryotic cell or a eukaryotic cell; and
incubating the prokaryotic cell or the eukaryotic cell so as to integrate the target DNA sequence into the target genome.

19. A cloning vector, being an autonomously replicating vector and containing: (a) one or more IIS type restriction endonuclease and/or meganuclease recognition sequences, and (b) multiple cloning sites.

20. The cloning vector according to claim 19, wherein the cloning vector is derived from: a pBR322 vector, a pUC vector, or a pET vector.

21. The cloning vector according to claim 19 or 20, containing two or more, for example, 3, 4, 5, 6, 7, 8, 9, 10 or more IIS type restriction endonuclease and/or meganuclease recognition sequences, wherein the IIS type restriction endonuclease recognition sequence is selected from any one or any combination of the following: recognition sequences of BbsI, BsaI, BsmBI, BspQI, BsrDI, EarI, HgaI and SfaNI.

22. (canceled)

23. The cloning vector according to claim 19, wherein the meganuclease recognition sequence is selected from any one or any combination of the following: recognition sequences of I-SceI, I-CreI, I-DmoI, I-OnuI, I-LtrI, I-PanMI, I-GzeMII, I-HjeI, I-LtrWI and I-SmaMI.

24. The cloning vector according to claim 19,
containing a replication origin and the selective marker gene or containing a lactose operon sequence, a β galactosidase encoding gene sequence containing the multiple cloning site, and 3 or more BspQI recognition sequences and/or 2 or more I-sceI recognition sequences.

25. (canceled)

26. The cloning vector according to claim 25, derived from the pUC vector.

27. (canceled)

28. The cloning vector according to claim 26 or 27, constructed by performing following reconstruction on the pUC57 vector: (i) the BspQI recognition sequence is added after a position 1554 base and a position 2539 base of the pUC57 vector and the I-sceI recognition sequence is added after a position 1501 base and a position 2479 base; and (ii) G at a position 1397 base of the pUC57 vector is mutated into C, and AT at a position 2136 base and a position 2137 base is mutated into GC.

29. The cloning vector according to claim 19, wherein its nucleotide sequence contains a sequence as shown in SEQ ID NO:1.

30. A kit for producing a target DNA sequence, containing: the cloning vector according to claim 19, protelomerase, one or more IIS type restriction endonucleases and/or meganucleases, and one or more exonucleases.
